(19) United States
(12) Patent Application Publication
(10) Pub. No.: US 2012/0269783 A1
(43) Pub. Date: Oct. 25, 2012

(54) COMPOSITION CONTAINING AUTOLOGOUS MONONUCLEAR CELLS AND PIG COLLAGEN SCAFFOLD IN PASTE FORM AND ITS USE IN THE PREPARATION OF A MEDICAMENT FOR SURGICAL TREATMENT

(75) Inventors: Sandro GIANNINI, Viareggio (IT); Roberto BUDA, Sasso Marconi (IT); Brunella GRIGOLO, Bologna (IT)

(73) Assignee: ISTITUTO ORTOPEDICO RIZZOLI, Bologna (IT)

(21) Appl. No.: 13/533,169

(22) Filed: Jun. 26, 2012

Related U.S. Application Data
(62) Division of application No. 12/530,081, filed on Sep. 4, 2009.

(30) Foreign Application Priority Data
Mar. 7, 2007 (IT) MI2007A000458

(51) Int. Cl. A61K 35/12; A61K 35/14; A61P 9/00; A61P 31/00; A61P 31/12; A61P 31/10; A61K 35/28; A61P 29/00

(52) U.S. Cl. 424/93.7

(57) ABSTRACT

Composition including autologous mononuclear cells supported on scaffold chosen from the group including lyophilized scaffold or pre-constituted scaffold in the form of membrane or tissue, with possible addition of platelet gel, as well as use of said composition for the preparation of medicaments for surgical treatments.

### TECHNICAL FIELD

The present invention concerns the treatment of osteochondral articular lesions.

Purpose of the repair of the articular cartilage is to restore the integrity of the joint surface, through the regeneration of hyaline cartilage. Hyaline cartilage can be obtained by methods that employ cell transplantations of chondrocytes (or their precursors).

### BACKGROUND ART

Nowadays, the state of the art involves the implant of autologous chondrocytes by arthroscopy. Such method entails a preliminary arthroscopy to harvest cartilage from the areas adjacent to the lesion or from a detached chondral fragment; the harvested chondrocytes are expanded in in vitro cell culture for a mean period of 5 weeks and seeded on biocompatible and bioabsorbable scaffolds based on hyaluronic acid, collagen or others; in a second arthroscopy the chondrocytes are implanted on scaffolds. This technique has the drawback of requiring two surgical sessions, the time for the necessary phase of in vitro cell expansion and the preparation of environments suitable for this purpose, high costs for the whole procedure, and great inconvenience for the patient. The characterizing limit of the employment of chondrocytes is the fact that they cannot be isolated if not in small quantities from cartilaginous tissue, and they require therefore a quantitative expansion in the laboratory. Such a process takes at least 15-20 days, and does not allow therefore the execution of a “one step” surgical technique.

Therefore, the need was particularly felt to overcome the drawbacks in terms of time, costs and inconvenience for the patient that the technique of transplantation of autologous chondrocytes involves.

### DISCLOSURE OF THE INVENTION

The applicants have surprisingly conceived and realized a biological material or composition including autologous mononuclear cells, more precisely, autologous mononuclear cells isolated from bone marrow, from peripheral blood or from fat tissue, supported by scaffold chosen from the group including lyophilized scaffold or pre-constituted scaffold in the form of membrane or tissue, with possible addition of platelet gel or other different signalling molecules. Said composition has been conceived for the preparation of medicaments for the treatment of osteocartilaginous articular lesions to be used in arthroscopic or arthrotomic surgery, in a single operating session.

According to the present invention it is possible to perform the full treatment in a single operating session, contrarily to the methods previously used that involve two surgical sessions.

Autologous mononuclear cells, according to the present invention, in fact, unlike chondrocytes, can be harvested in great quantities and packed, directly during the operating session.

In the orthopedic field its usefulness is known in the treatment of nonunions (Hernigou, et al.: J Bone Joint Surg Br 2005 July; 87 (7): 896-902) and necroses of the femoral head (Gangji, et al.: J Bone Joint Surg Am. 2004 June; 86-A (6): 1153-60). To be implanted in great number in the lesion area, such cells need a scaffold on which to be seeded.

The scaffolds that belong to the composition of the present invention, can be lyophilized scaffolds, particularly collagen of pig origin (such as Spongostan® Powder or Surgiflo®). This is a bioabsorbable and biocompatible material already broadly used in surgery to provide surgical haemostasis, which acts well as a malleable scaffold to deliver the cells to the site of the lesion, but never indicated as possible scaffold to be combined with autologous mononuclear cells, preferably those isolated from bone marrow, peripheral blood or fat.

The composition of the present invention, besides its employment in the treatment of osteocartilaginous articular injuries in a single operating session in arthroscopic or arthrotomic surgery, allows the aforementioned drawbacks of the autologous chondrocyte transplantation technique to be overcome.

One object of the present invention consists of a biological material or composition including autologous mononuclear cells supported on scaffold chosen from the group including lyophilized scaffold or pre-constituted scaffold in the form of membrane or tissue.

In a preferred form of realization of the composition of the present invention, the autologous mononuclear cells used are autologous mononuclear cells of medullary origin, particularly those harvested from the posterior iliac crest, or autologous mononuclear cells isolated from peripheral blood or from fat.

In a further even more preferred form of realization of the composition according to the invention, the scaffold supporting the autologous mononuclear cells, more precisely those of medullary origin, chosen from the group including lyophilized scaffold or pre-constituted scaffold in the form of membrane or tissue, is a biocompatible and bioabsorbable scaffold and can be constituted by various composite materials, such as natural, semi-synthetic or synthetic polymers.

The natural polymers used for the invention are selected from the group including collagen, collagen and glycosaminoglycan co-precipitates, hyaluronic acid, cellulose, polysaccharides in the form of chitin gel, chitosan, pectin or pectic acid, agar, agarose, xanthan, gellan gum, alginic acid or alginates, polymannans or polyglycans, starch and natural rubbers, and demineralized bone matrix (DBM).

The semi-synthetic polymers used for the invention are selected from the group including reticulated collagen with agents such as aldehydes or their precursors, dicarboxylic acids or their halides, diammine, derivates of cellulose, hyaluronic acid, chitin gel or chitosan, gellan gum, xanthan, pectin or pectic acid, polyglycans, polymannans, agar, agarose, natural rubber and glycosaminoglycan.

The useful synthetic polymers to achieve the invention are selected from the group including polylactic acid, polyglycolic acid or their copolymers or derivates, polydioxane, polyphosphazenes, polysulfonic resins, polyurethanes and PTFE, hydroxyapatite in paste form.

In a preferred realization of the invention the composition includes a scaffold constituted by pig collagen.

In addition, it has been found that various signalling molecules, including growth factors, can be added to the composition of the present invention to favor the multiplication and the differentiation of the autologous mononuclear cells, particularly those from packed bone marrow.

In a further preferred form of realization of the composition of the present invention, these growth factors are provided platelet gel is obtained by harvesting venous blood the day before the operation, through an automated procedure, such as Vivostat®.

The platelet gel according to the invention can be placed on the surface of the biological material or composition of the present invention or on the bed of the lesion or in the composition of the paste.

The biological material or composition of the present invention can also include pharmacologically or biologically active substances. These pharmacologically and biologically active substances are selected from the group including anti-inflammatory agents, antibiotics, growth factors, and antimycotic and antiviral agents.

Another object of the present invention is the use of biological material or composition, in any of the forms described above, for the preparation of a medicament for the treatment of articular osteocartilaginous injuries in a single operating session in arthroscopic or arthrotomic surgery.

In a preferred form of realization of the present invention articular osteocartilaginous lesions are ostoarticular lesions of knees and ankles.

According to the present invention it has also been found that it is possible to preserve, in a preferred form cryopreserve, preventively the biological material or composition, according to any of the forms of preferred realization described above, to store its characteristics of cellular viability for grafts in subsequent arthroscopic or arthrotomic procedures.

Another object of the present invention is a method of treatment of articular osteocartilaginous lesions in a single operating session by the implant in arthroscopic or arthrotomic surgery of a biological material or composition including autologous mononuclear cells and a scaffold chosen by the group including lyophilized scaffold or pre-constituted scaffold in the form of membrane or tissue, said method including:
Drawing of medullary blood from the posterior iliac crest during the operating session;
Preparation of a concentrate of autologous mononuclear cells from Medullary aspirate with an automated method of centrifugation in the operating room, (such as the BMAC Harvest® method or others);
Arthroscopy or arthrotomy of the joint to be treated;
Arthroscopic or arthrotomic cleaning of the lesion and relief of combined conditions;
Preparation of a composition including the concentration of autologous mononuclear cells from medullary aspirate and scaffold of support chosen by the group including lyophilized scaffold or pre-constituted scaffold in the form of membrane, said composition obtained for mixing said components together;
Implant of the composition to fill the center of infection of the osteochondral lesion in arthroscopy or arthrotomy.

In a preferred form of the method of treatment of articular osteocartilaginous injuries in a single operating session according to the present invention, the lyophilized scaffold is pig collagen (such as Spongostan° Powder or) Surgiflo®.

In a further preferred form of realization of the method of treatment of articular osteocartilaginous injuries in a single operating session according to the present invention, said method also includes before the phase of drawing of the medullary blood from the iliac crest the following phases:
Drawing of venous blood the day before the operation;
Preparation of platelet gel with automated procedure, such as the Vivostat® procedure;
freezing of the platelet gel up to the moment of the operation;

from which the preserved platelet gel is, alternatively:

1) mixed in the composition including the concentration of autologous mononuclear cells from medullary aspirate and support scaffold; or

2) placed in the interface between the composition including the concentration of autologous mononuclear cells from medullary aspirate and support scaffold and the bed of the osteo-chondral lesion; or

3) spread on the implant of the composition including the concentration of autologous mononuclear cells from medullary aspirate and support scaffold, the implant in turn is spread on the center of infection of the osteoarticular lesion; or

4) mixed in the composition according to point 1) and situated in the interface according to point 2); or

5) mixed in the composition according to point 1) and spread on the implant according to point 3); or

6) placed on the interface according to point 2) and spread on the implant according to point 3); or

7) contemporarily present in the composition according to point 1), placed on the interface according to point 2) and spread on the implant according to point 3).

The rationale of the method of treatment of articular osteocartilaginous injuries in a single operating session, according to the present invention, includes as a further preferred form:
the drawing of venous blood the day before the operation for the preparation of the platelet gel with automated method, such as Vivostat® with possible freezing of the obtained platelet gel (these phases in optional form);
in the operating site a medullary aspirate from the posterior iliac crest is performed, it is concentrated in the operating room by an automated method of centrifugation, with the use for instance of the Harvest device to get a concentration of mononuclear cells;
during the concentration of the cells an arthroscopy or arthrotomy is performed of the joint to be treated with relief and recovery of the osteochondral lesion;
the medullary concentrate is used for preparing a composition, preferably in paste form, by mixing with the collagen of pig origin (such as Spongostan® Powder or surgiflo®);
the composition or paste is subsequently inserted by arthroscopy or arthrotomy to fill the site of the lesion;
finally, optionally, a layer of platelet gel is spread onto it or, alternatively, the platelet gel can be placed on the bed of the lesion or in the composition or paste when the prepared platelet gel is used in the optional phases.

A further object of the present invention includes the process for the preparation of the composition, in any of the preferred forms of realization described above, said process includes:
Preparation of a concentration of autologous mononuclear cells from medullary aspirate with an automated method of centrifugation in the operating room, such as the BMAC® Harvest method;
Preparation of an composition including the concentrate of autologous mononuclear cells from medullary aspirate and support scaffold chosen by the group including lyophilized scaffold or pre-constituted scaffold in the form of membrane or tissue, said composition obtained for mixing said components together.
   Optionally said process of preparation includes in the phase of preparation of the composition the mixing of platelet gel obtained by the automated procedure with venous blood, or having obtained the composition of autologous. mononuclear cells and scaffold, the distribution around the said composition, in contact with it, of platelet gel, preferably in layer form, obtained with the automated procedure from venous blood.

1. A composition including autologous mononuclear cells, a scaffold for supporting said cells in the form of a membrane or a tissue and a platelet gel.

2. The composition according to claim 1, wherein the cells are selected from: autologous mononuclear cells isolated from bone marrow, autologous mononuclear cells isolated from peripheral blood and autologous mononuclear cells isolated from fat.

3. The composition according to claim 2, wherein the cells are autologous mononuclear cells isolated from bone marrow.

4. The composition according to claim 3, wherein the cells are withdrawn from the posterior iliac crest.

5. The composition according to claim 1, wherein the scaffold is lyophilized scaffold.

6. The composition according to claim 1, wherein the scaffold comprises a natural polymer, or a semi-synthetic polymer, or a synthetic polymer.

7. The composition according to claim 6, wherein the natural polymer is selected from: collagen, a co-precipitate of collagen and glycosaminoglycan, hyaluronic acid, a cellulose, a polysaccharide in the form of chitin gel, a chitosan, a pectin or pectic acid, an agar, an agarose, a xanthan, a gellan gum, an alginic acid or an alginate, a polymannan or a polyglycan, a starch and a natural rubber, and a demineralized bone matrix (DBM).

8. The composition according to claim 7, wherein the scaffold is collagen of pig origin.

9. The composition according to claim 6, wherein the semi-synthetic polymer is selected from: a reticulated collagen with an agent such as an aldehyde or its precursor, a dicarboxylic acid or its halide, a diammine, a derivate of cellulose, a hyaluronic acid, a chitin gel or a chitosan, a gellan gum, a xanthan, a pectin or a pectic acid, a polyglycan, a polymannan, an agar, an agarose, a natural rubber and a glycosaminoglycan.

10. The composition according to claim 6, wherein the semi-synthetic polymer is selected from: a polylactic acid, a polyglycolic acid or its copolymer or derivate, a polydioxane, a polyphosphazene, a polysulfonic resin, a polyurethane and PTEE, hydroxyapatite in paste or tissue form.

11. The composition according to claim 1, also including a pharmacologically or biologically active substance.

12. The composition according to claim 11, wherein the pharmacologically and biologically active substance is selected from: an anti-inflammatory agent, an antibiotic, a growth factor, an antimycotic and an antiviral agent.

13. The composition according to claim 1, in which the composition is preserved for a subsequent graft.

14. The composition according to claim 1, in which the composition is cryopreserved.

15. A process for the preparation of a composition according to claim 1, said process comprising the following steps:
(i) performing a bone marrow aspiration in an individual and obtaining a bone marrow aspirate;
(ii) preparing a concentrate of autologous mononuclear cells from the bone marrow aspirate by automated centrifugation; and
(iii) preparing a composition by mixing the concentrated cells according to step (ii), a platelet gel and a scaffold for supporting said cells.

16. The method according to claim 15, wherein the platelet gel according to step (iii) is prepared before performing step (i).

17. The method according to claim 16, wherein the platelet gel is prepared according to the following steps:
(iv) obtaining a venous blood sample from an individual;
(v) preparing a platelet gel with automated procedure from the blood sample according to step (iv); and
(vi) freezing the platelet gel of step (v) up to the moment of use.

18. A method for the treatment of articular osteocartilaginous injuries in arthroscopic surgery in a single operation session, comprising administering the composition of claim 1 to a patient in need thereof.

19. A method for the treatment of articular osteocartilaginous injuries in arthrotomic surgery in a single operation session, comprising administering the composition of claim 1 to a patient in need thereof.
